(19) United States
(12) Patent Application Publication
(10) Pub. No.: US 2009/0238888 A1
(43) Pub. Date: Sep. 24, 2009

(54) RADICAL POLYMERIZATION METHOD AND PRODUCTS PREPARED THEREBY

(75) Inventor: Jun Hu, Fairlawn, OH (US)

Correspondence Address: ROETZEL AND ANDRESS, 222 SOUTH MAIN STREET, AKRON, OH, 44308, US

(73) Assignee: THE UNIVERSITY OF AKRON, Akron, OH (US)

(21) Appl. No.: 11/577,847

(22) PCT Filed: Oct. 31, 2005
(86) PCT No.: PCT/US05/39084

§ 371 Date: Jun. 2, 2009

Related U.S. Application Data
(60) Provisional application No. 60/623,657, filed on Oct. 29, 2004.

(51) Int. Cl. C08F 2/32; A61K 9/50; C12N 9/00; C12N 5/00; C08L 31/02; H01F 1/26

(52) U.S. Cl. 424/497; 526/217; 435/183; 435/325; 524/556; 524/543; 252/62.54

(57) ABSTRACT

In one embodiment, the present invention relates to a method for initiating radical polymerization of at least one monomer composition, the method comprising the steps of: supplying at least one monomer charge; and initiating radical polymerization of the at least one monomer charge via a hydrogen peroxide initiator and at least one polyamine co-initiator, wherein the method is carried out in an inverse-microemulsion, the inverse-microemulsion being a water/oil emulsion.

### FIELD OF THE INVENTION

The present invention relates to a method for producing polymer macromolecules. And macromolecule compounds produced by the methods disclosed herein. More specifically, the present invention relates to methods for producing amphiphilic comb polymers using an inverse emulsion radical polymerization scheme. In one embodiment, an inverse emulsion radical polymerization scheme according to the present invention utilizes at least one waterborne macro-initiator in combination with at least one oil soluble monomer to produce a polymer macromolecule compound.

### BACKGROUND OF THE INVENTION

Recently, polymer nanocapsules have attracted a great deal of interest in the development of new intracellular delivery systems such as delivery systems for biomedical imaging, targeted drug delivery, and gene therapy. Polymer materials exhibit a range of supramolecular structures and functionalities, which could potentially allow for chemical tailoring of material properties for target-specific applications.

Recent advances in the synthesis of macromolecules have permitted, among other things, the preparation of novel materials with nanoscopic architectures that arise from self-assembly of the polymer domain structures. These materials can be used, for example, to prepare polymer nanocapsules.

Amphiphilic comb polymers are one of the complex macromolecular architectures that have attracted attention due to their unique supramolecular behaviors at the water-oil interface. Several methods have been developed for comb polymers by co-polymerization of long chain monomers (macromonomers) or by condensation excess of reactive long chains with a multifunctional backbone polymer. Although cationic, anionic, and condensation polymerization methods have been used, controlled radical polymerizations, such as reversible addition-fragmentation chain transfer polymerization (RAFT) and atom transfer radical polymerization (ATRP), are widely used. To date, the amphiphilic nature of a comb polymer has not been exploited in the preparation of such materials using radical polymerization synthesis routes. Accordingly, there is a need in the art for improved synthesis routes for polymer macromolecules, and in particular amphiphilic comb polymers.

### SUMMARY OF THE INVENTION

The present invention relates to a method for producing polymer macromolecules. And macromolecule compounds produced by the methods disclosed herein. More specifically, the present invention relates to methods for producing amphiphilic comb polymers using an inverse emulsion radical polymerization scheme. In one embodiment, an inverse emulsion radical polymerization scheme according to the present invention utilizes at least one waterborne macro-initiator in combination with at least one oil soluble monomer to produce a polymer macromolecule compound.

In one embodiment, the present invention relates to a method for initiating radical polymerization of at least one monomer composition, the method comprising the steps of: supplying at least one monomer charge; and initiating radical polymerization of the at least one monomer charge via a hydrogen peroxide initiator and at least one polyamine co-initiator, wherein the method is carried out in an inverse-microemulsion, the inverse-microemulsion being a water/oil emulsion.

In another embodiment, the present invention relates to a radical-initiator polymerization system comprising: at least one monomer; a hydrogen peroxide initiator; and at least one co-initiator, wherein the at least one co-initiator is a polyamine co-initiator and wherein polymerization is carried out in an inverse-microemulsion, the inverse-microemulsion being a water/oil emulsion.

### BRIEF DESCRIPTION OF THE DRAWINGS

FIG. 1 illustrates different types of structures that can be produced via the radical polymerization method of the present invention;

FIG. 2(a) is a ¹H NMR spectra of an amphiphilic graft copolymer made by a process according to one embodiment of the present invention;

FIG. 2(b) is a ¹³C NMR spectra of the amphiphilic graft copolymer of FIG. 2(a);

FIG. 3 is an FT-IR spectra of the polymer of FIGS. 2(a) and 2(b);

FIG. 4 is a DSC thermogram of polyallylamine showing a T_{g} around −6° C. and a DSC thermogram of one core-shell material according to the present invention with two T_{g}'s around −1° C. and 55° C., representing two phases;

FIG. 5 illustrates the formation of a core-shell material in accordance with one embodiment of the present invention;

FIG. 6 is a TGA thermogram of polyallylamine illustrating one decomposition temperature and a TGA thermogram of the core-shell material illustrating two decomposition temperatures;

FIG. 7 shows SEM micrographs of a core-shell material according to one embodiment of the present invention; and

FIG. 8 shows TEM images of a core-shell material according to one embodiment of the present invention.

### DETAILED DESCRIPTION OF THE INVENTION

The present invention relates to a method for producing polymer macromolecules. And macromolecule compounds produced by the methods disclosed herein. More specifically, the present invention relates to methods for producing amphiphilic comb polymers using an inverse emulsion radical polymerization scheme. In one embodiment, an inverse emulsion radical polymerization scheme according to the present invention utilizes at least one waterborne macro-initiator in combination with at least one oil soluble monomer to produce a polymer macromolecule compound.

In one embodiment, the present invention relates to amphiphilic core-shell materials produced by a graft copolymerization technique through inverse emulsion polymerization. In one instance, the process of the present invention can produce the above-mentioned product in a one-step process. In one embodiment, amphiphilic core-shell materials in accordance with the present invention have a core-shell structure with a hollow and functionalized aqueous core within a cross-linked hydrophobic shell. In this embodiment, the core structure has pendant free amine groups that swell in water. The hydrophobic shell is “grafted to” the hydrophilic core by copolymerizing a hydrophobic monomer from a water-soluble amine polymer. The hydrophobic shells are generally cross-linked to some extent in order to maintain the structural integrity of the core-shell polymer assembly.

The approached used in the present invention, is based on the generation of an initiating radical on a water soluble amine polymer in an aqueous phase of the inverse emulsion, by a water soluble initiator at the water/oil interface, and subsequent grafting of, for example, a vinyl monomer to the amine polymer from the interface. The graft copolymer, thus generated can be further manipulated to yield a core-shell structure. The ultimate structure is a hollow aqueous core with one or more functionalized amine groups inside a partially cross-linked acrylate shell. This structure, among other things, allows for tremendous flexibility in the encapsulation of hydrophilic drugs, cells, enzymes and other biomaterials. Apart from biomedical applications these materials can also be used as sensing materials, catalysis, coating materials, filtration units, etc.

A general scheme for forming amphiphilic structures via an inverse emulsion polymerization route, in accordance with the present invention, is shown schematically below:

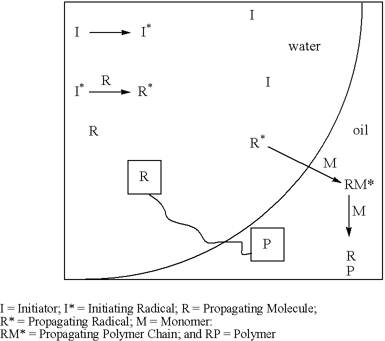

A variety of emulsion and emulsion polymerization schemes exist, and will be explained in more detail below. In standard emulsion polymerization system the monomers (oil soluble) cluster into micelles in the dispersed water phase in presence of a surfactant beyond the critical micelle concentration (CMC). This is typically referred as oil in water emulsion. Upon the addition of the initiator, either water soluble or oil soluble, radicals are generated which form oligoradicals in the dispersed phase. These oligoradicals then migrate into the monomer swollen micelles to propagate the polymerization reaction. The monomer swollen particles become the main site for polymerization reaction. The monomer required for the polymerization is continuously supplied from the monomer droplets by diffusion through the dispersed phase. The polymerization is completed when all monomer is converted into polymer. The final polymer is in the form of particle dispersed in the water phase stabilized by the adsorbed surfactant molecules.

The role of surfactant (also known as emulsifier) molecules is very important in stabilizing the emulsion systems and the polymer colloids formed thereby. Hydrophilic lipophilic balance (HLB) values are determined on the basis of the solubility parameters (δ²) of the hydrophilic and lipophilic end of the surfactant molecules based on the cohesive energy ratio (CER) concept. Typically emulsifiers with HLB value greater than 7 promote oil in water (O/W) emulsion and those with HLB value less than 7 promote water in oil (W/O) emulsion. The W/O emulsion are generally referred as inverse or reverse emulsion as compared to the standard oil in water emulsion. The stability and the size of the emulsion are also controlled by the concentration of the emulsifier molecules in the system. In terms of stability and size, emulsions are classified as macro-, mini-, and micro-emulsions. With the decreasing order of size in the emulsion the stability increases.

Macroemulsions with droplet sizes of about 1 to about 100 μm are the least stable, while microemulsions with droplet sizes of about 10 to about 100 nm are the most stable. Miniemulsions have intermediate stability and have droplet sizes ranging from about 50 to about 500 nm. Typically smaller emulsions are stabilized via an increased concentration of emulsifier (or a mixture of emulsifiers) in the presence of one or more co-surfactants and/or stabilizers. The chart below points out the key parameters that determine the nature of the emulsion.

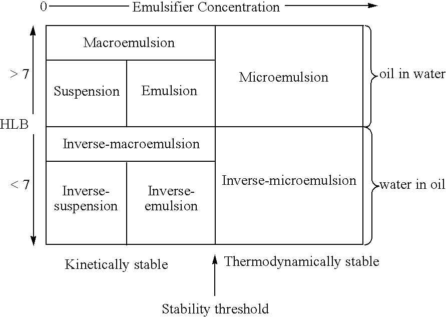

Polymerization in an inverse emulsion is usually more complex than conventional emulsion polymerization processes. Water in oil (W/O) emulsion systems are formed with an emulsifier of HLB value less than 7. The inverse emulsions are usually less stable than their standard counterpart due to electrostatic factors in addition to the steric factors, which are predominant in standard emulsions. An increased concentration of emulsifier is necessary to increase the stability of such systems in addition to the presence of additional emulsifiers (co-emulsifier) and stabilizers. Due to the inherent nature of inverse emulsion, such systems sometime yield bi-continuous phase products instead of globular structures. Both oil soluble and water soluble initiators can be used to initiate the polymerization reaction of the present invention depending various parameters of the polymerization systems disclosed herein.

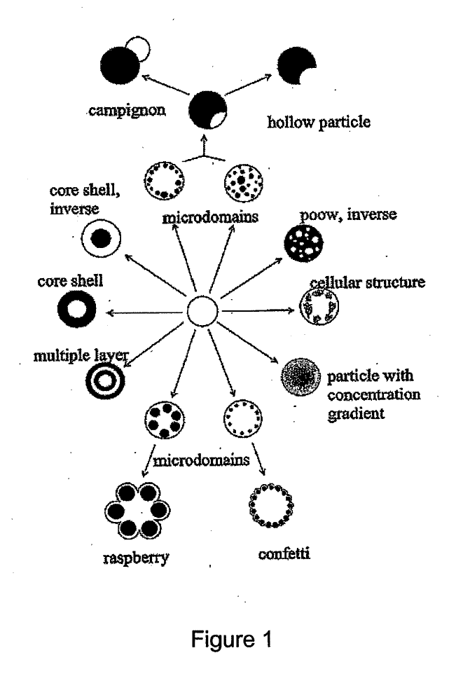

Emulsion and inverse polymerization systems generate different structural features depending on the reaction parameters and other factors. FIG. 1 reveals various possible structures that can be generated using emulsion polymerization schemes. However several important factors play an important role in the formation of a core-shell morphology. While not wishing to be bound to any one theory, in general core-shell morphology is preferred when radicals are generated at the interface of the two immiscible (water and oil) phases and if diffusion is restricted to the interior.

### Materials:

t-Butyl acrylate (t-BA) (Aldrich, 98%) and ethylene diacrylate (ED) (Acros, 70%) are washed with 5% NaOH aqueous solutions (Merck, 97%), dried over anhydrous MgSO₄ (Fischer, 99%) overnight, and stored at −12° C. prior to use. Polyallylamine, (PAA, a 20 weight percent solution in water, M_{w} 17,000, Aldrich), H₂O₂, (a 30 weight percent solution in water, Aldrich), toluene (Fischer, 99.9%), methylene chloride (Fischer 99.9%), and sorbitan monooleate (Span® 80, Aldrich) are used as received from their suppliers. The water needed for the present invention is distilled water (dH₂O).

Polyallylamine (PAA) is chosen as the water soluble polymer to graft the hydrophobic acrylate monomer to the backbone of the PAA. It should be noted that the present invention is not limited to the above combination of PAA and acrylate monomer. Rather, any suitable water-soluble polymer can be used so long as the polymer chosen is receptive of/able to have a hydrophobic monomer grafted to its polymer backbone. Suitable hydrophobic monomers include, but are not limited to, acrylate monomers, vinyl alcohol monomers, vinyl ether monomers, and carbonate monomers. Suitable water-soluble polymers that can be used in the present invention include, but are not limited to, polyallylamines, polyamines, polyamides, and biodegradable polymers that contain at least one amino acid functionality. The main reason PAA is chosen is due to its high water solubility, presence of primary amine group in the repeating units and its commercial availability. The two protons present on the amine group in the repeating units offer enough sites to initiate the radical polymerization by a peroxide initiating system. The second component of the system is ter-Butyl acrylate (t-BA) monomer. This monomer has several biological applications and the presence of ter-butyl group in the repeating units of the grafted chain provides opportunity for further chemical modification by easy and facile deprotection of the ter-butyl group of the ester. The chemical structures of the above compounds are shown below:

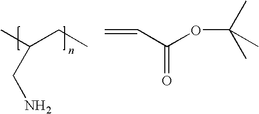

where n is an integer from about 10 to about 10,000; or from about 25 to about 5,000; or from about 50 to about 2,500; or from about 100 to about 1,000; or even from about 250 to about 500. Here, as well as elsewhere in the specification, ranges can be combined.

The initiator used in the polymerization reactions of the present invention is chosen to be water soluble so that the initiating radical generates the radical propagation site on the water soluble polymer (i.e., the polyallylamine) present in the water phase. Commonly used water soluble initiators such as potassium and ammonium persulfates (S₂O₈⁻²) were avoided in this specific instance due to strong oxidizing capacity of the persulfate ion which could oxidize the primary amine group of the polymer chain. Highly water soluble t-butyl hydroperoxide (TBHP) is not used in this instance because of its tendency to generate radical in the oil phase in spite of its high water solubility. Moreover, the initiation of TBHP would leave toxic residue which could be detrimental to any biological application of the polymer. Considering all the facts, hydrogen peroxide (H₂O₂) is used as initiator to generate a radical on the nitrogen through the abstraction of protons from the amine group (see the scheme below for more detail). However the presence of multi-valent cations (Mⁿ⁺—for example, Fe²⁺), usually present in the water phase in the parts per million level might/can catalyze the initiation in amine/peroxide system.

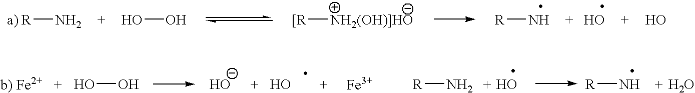

Generation of initiating radical in absence and presence of metal catalyst

In one embodiment, the emulsifier of the present invention is chosen so as to generate a water in oil (W/O) emulsion. As discussed above, emulsifiers with a HLB value lower than 7 generate W/O emulsions. Sorbitan monooleate (Spano® 80), as shown below, with HLB value 4.3 is used to create an inverse water in oil emulsion.

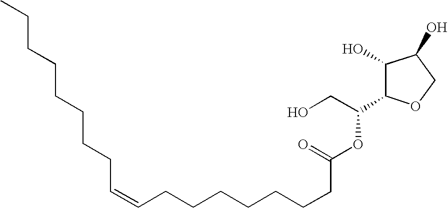

In this embodiment, additional co-surfactants and/or stabilizer molecules were not used on the basis that the radicals generated on the polyallylamine polymer would serve to provide extra stability in addition to that provided by the Span® 80. Thus, in this embodiment the polyallylamine macroradical serves the dual purpose as both the site of propagation, and as a stabilizer to the inverse emulsion. The emulsifier behavior of the polyallylamine macroradical is due to the presence of hydrophilic amine groups and the hydrophobic hydrocarbon backbone of the polymer.

### Preparation of W/O Emulsion:

A three-necked round bottom flask is equipped with a thermometer, a condenser, an argon inlet and a magnetic stir bar. The flask is charged with PAA (0.2 g in 5 mL of water), t-BA monomer (4.5 mL), ethylene diacrylate (0.7 mL in 5 mL toluene), H₂O₂ (1.0 mL), and Span® 80 (0.17 g). The resulting mixture is sonicated (Fisher FS6) at room temperature and at approximately 400 rpm for 30 minutes to produce an inverse emulsion which is stable for at least stable 20 minutes when left sifting at room temperature. The exact recipe of the inverse emulsion is given in Table 1. The emulsion was also stable without ethylene diacrylate, and showed no phase separation during a period of about 20 minutes at room temperature.

TABLE 1

| Ingredients | Amount | |
|---|---|---|
| Water Phase | | |
| Polyallylamine | 0.2 | g (20% wt. soln.) |
| H₂O₂ | 1.0 | ml (30% wt. soln.) |
| H₂O | 5.0 | ml |
| Oil Phase | | |
| t-butyl acrylate | 4.50 | ml |
| Ethylene diacrylate | 0.70 | ml |
| Toluene | 5.0 | ml |
| Surfactant | | |
| Span ® 80 | 0.17 | g |

### Synthesis of Amphiphilic Graft Copolymer:

An emulsion polymerization mixture is prepared according to the above recipe except that no cross linker ethylene diacrylate is added. The round bottom flask is placed in an oil bath and was heated at 65° C.±2° C. for 4 hours under constant argon purge with uniform stirring with a magnetic stirrer. After the above reaction is complete, 50% w/v ethanol (30 mL) is added to the reaction mixture to precipitate the polymer. A yellowish sticky solid is obtained after removing the solvents and dried under the vacuum at room temperature (yield 0.7 gm).

### Synthesis of Core-Shell Material:

An emulsion polymerization mixture is prepared according to the above recipe in Table 1. The reaction mixture is heated at 65° C.±2° C. for 4 hours under constant argon purge with uniform stirring. The resulting milky suspension is diluted with water and stirred vigorously such that there is no aggregate in the mixture. The polymer is then colleted by filtration and dried under vacuum at room temperature to yield a light yellow solid polymer (yield 1.0 g). The resulting product is then washed with methylene chloride to remove any residual unreacted monomer and other impurities. The final product is re-dispersed in water and stored at room temperature over a period of several weeks during which tests are conducted on this product to determine its chemical properties and polymer structure. Also, the product is subjected to morphological analyses. During this time, no significant changes in the appearance of the material is observed.

### Characterization of Graft Copolymer and Core-Shell Polymer:

The grafting density and efficiency of the copolymerization were analyzed according to the standard methods (see Athawale et al.; Carbohydr. Polym., 2000, Vol. 41, p. 407 and Chun et al.; J. Appl. Polym. Sci, 1997, Vol. 64, p. 1733) and the results are shown in Table 2 below. The copolymer product is washed with methylene chloride and water, and is dried overnight under vacuum at room temperature to obtain a solid polymer particle. The grafting characteristics, reproducible within ±10%, are expressed by the following equations:

$$\begin{matrix}Grafting Percentage ( \% ) = \frac{{w}_{2} - {w}_{1}}{{w}_{1}} \times 100 \\ Grafting Efficiency ( \% ) = \frac{{w}_{2} - {w}_{1}}{{w}_{3}} \times 100 \\ Grafting Ratio ( \% ) = \frac{{w}_{1} - {w}_{4}}{{w}_{1}} \times 100\end{matrix}$$

where, w₁, w₂, w₃ and w₄ are the weight of PAA, grafted-PAA, monomer and non-grafted PAA, respectively.

TABLE 2

| | Grafting | Grafting | Grafting |
|---|---|---|---|
| Material | Percentage (%) | Efficiency (%) | Ratio (%) |
| t-butyl acrylate grafted | 239 | 24.5 | 88.5 |
| polyallylamine | | | |

### Characterization Methods:

NMR analysis is performed with a Varian Gemini 300 NMR spectrometer and FT-IR analysis is performed with a Nicolet NEXUS 870 FT spectrometer. For NMR characterizations for the polyallylamine-g-poly(t-butyl acrylate) comb polymer, CDCl₃ is used as the solvent and internal reference (7.20 ppm for ¹HNMR and 77.76 ppm for ¹³CMR). The shell cross-linked polymeric nanocapsules are insoluble in most NMR solvents.

Differential scanning calorimetry studies of the solid polymer samples are performed with a DSO Q100V7.0 Build 244 (Universal V3. 7A TA) instrument at a scanning rate of 10° C./min. Thermo gravimetric analysis (TGA) is performed with a TGA Q50V5.0 Build 164 (Universal V3. 7A TA) instrument. Scanning electron microscopy (SEM) studies are performed with a Hitachi S2150 instrument with an operating voltage of 15 kV. Transmission electron micrograph (TEM) studies are performed with a Jeol Transmission Electron Microscope (1200 EX II) at accelerating voltage of 120 kV.

### Results and Discussion:

The present invention is, in one embodiment, based on the generation of the initiating radical on a water soluble polyallylamine backbone exclusively in the aqueous phase of an inverse microemulsion. Thus, radical polymerization is confined at the water/oil interface with an oil soluble t-butyl acrylate. The radical chain propagated into the oil phase while the polyallylamine backbone remains in the water phase (see Scheme 1 above). Subsequent rearrangement of the nitrogen centered radical to the carbon backbone of the polymer is thermodynamically favorable (see Scheme 2 below).

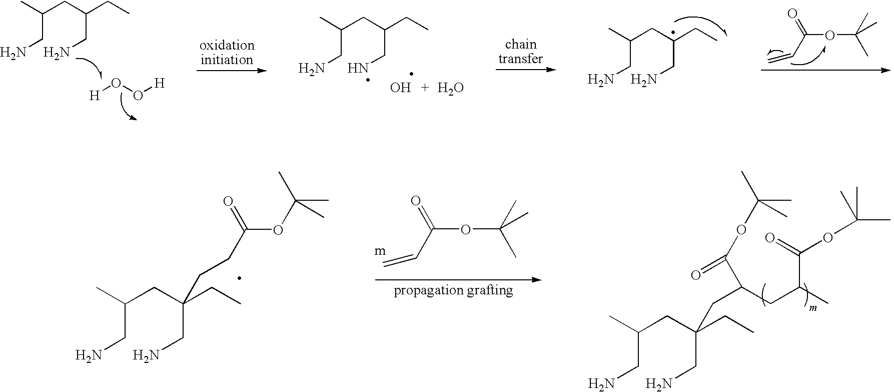

This process may be catalyzed by traces of metal ions such as iron in the reaction mixture and may be inhibited by acids that protonate the basic amine group.

To develop a waterborne radical macroinitiator, one needs to examine the reactions between polyallylamine and several oxidation agents including t-butyl hydrogen peroxide and potassium persulfate. As a result of the present invention, it can be stated that a combination of hydrogen peroxide (30% aqueous solution) and polyallylamine initiated the radical polymerization smoothly in a water in oil microemulsion. The inverse microemulsions are produced by using Span® 80 (HLB=4.3) as the main emulsifier. To obtain inverse microemulsions which are comparatively less stable than oil in water (O/W) emulsions, it is necessary to use a high concentration of emulsifiers having HLB value less than 7.

Electrostatic and/or steric factors are also important for the stability of the system. In the case of the present invention, it is believed hat the amphiphilic comb polymer itself also serves as a surfactant during the polymerization with the polyallylamine acting as the aqueous soluble segment and poly t-butylacrylate as the hydrophobic segment. The unique comb polymer architecture of the polyallylamine-g-poly(t-butyl acrylate) of the present invention provided additional stability to the microemulsion and ensured the radical chains only initiated on the water soluble polyallylamine backbone.

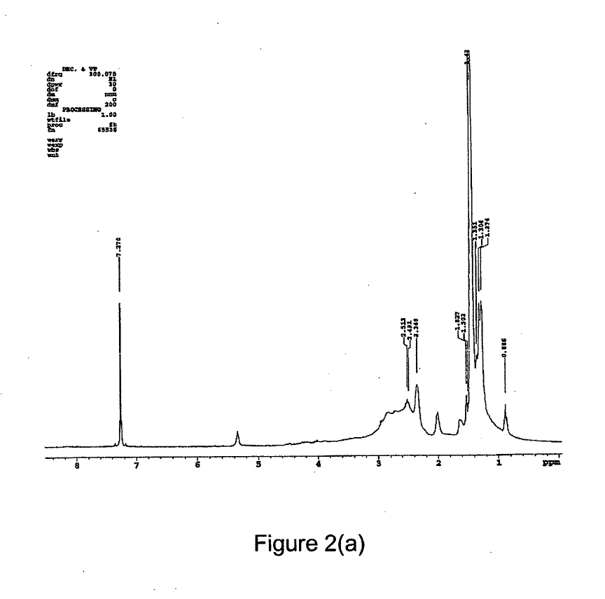

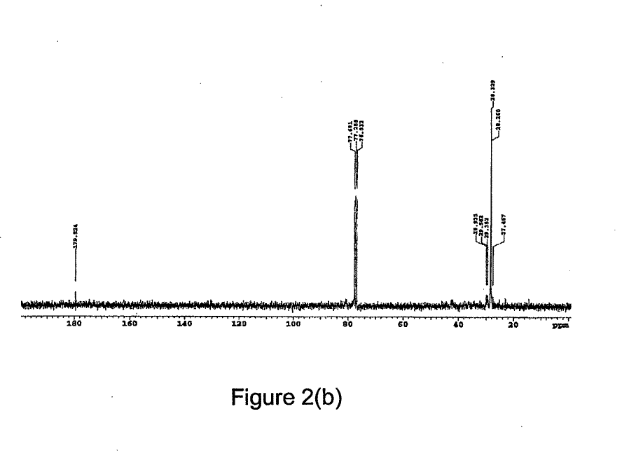

The initial experimental evidence for the formation of polyallylamine-g-poly(t-butyl acrylate) is from the solution ¹H and ¹³C NMR spectra of the copolymer as shown in FIG. 2(a). ¹H NMR: δ 0.8-1.8 (polymer backbone, —CH₂—C—), 1.4 (t-butyl group, —C(CH₃)₃), 1.5 (acrylate chain, —CH₂—), 2.0 (acrylate chain, —CH— and amine, C—NH₂), 2.5 (methylene group in polyallylamine, —CH₂—N). ¹³C NMR: δ 29.3 (t-butyl group, —C(CH₃)₃), 40.0 (methylene group in polyallylamine —CH₂—N), 37.0-39.0 (methyne carbons), 179.3 (carbonyl group). These results are consistent with the proposed polyallylamine-g-poly(t-butyl acrylate) copolymer architecture.

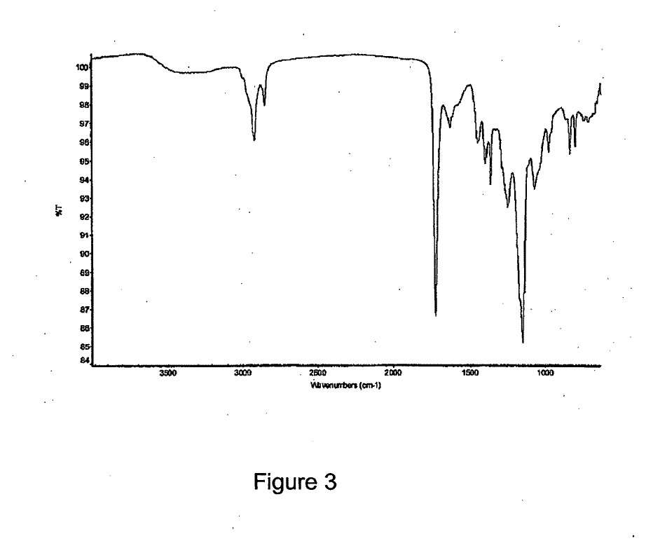

IR spectrum of the copolymer confirms the NMR results (see FIG. 3). The strong band at 1728 cm⁻¹ is assigned to the carbonyl group stretching of the acrylate ester, which indicates the presence of t-BA in polymer. The broad band at 3200-3450 cm⁻¹ is assigned to the N—H stretching and the small band at 1637 cm⁻¹ is assigned to N—H waiving mode. Both of these N—H peaks were present in the polyallylamine spectrum which indicated the PAA back bone remained in the copolymer product.

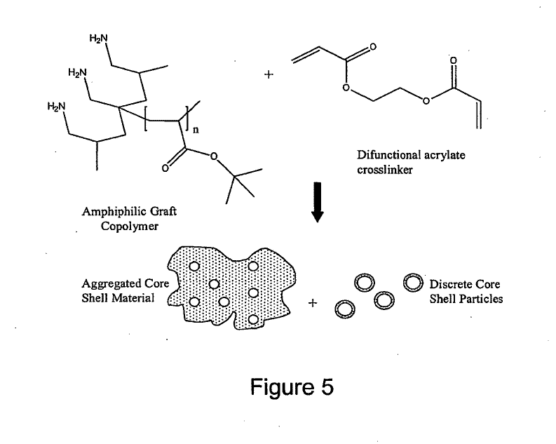

It is well known to those of ordinary skill in the art that the core-shell morphology of a polymer produced in an emulsion is preferred only when the initiating radicals are formed at the water/oil interface and its diffusion to the interior of the emulsion is restricted. Accordingly, examination of the formation of microcapsules by in situ cross-linking of the amphiphilic comb polymer is conducted with the dual objective of: (1) verifying the interfacial polymerization mechanism for the comb polymer synthesis, and (2) developing new methods of preparing shell cross-linked amphiphilic polymer nanocapsules (see Scheme 3 in FIG. 5 which illustrates the formation of a core-shell material).

The copolymer produced with the cross-linker shows limited solubility in organic solvents, which differs substantially from the corresponding uncross-linked copolymer (see Table 3).

TABLE 3

| | Time | | | |
|---|---|---|---|---|
| Solvent | 5 Hours | 10 Hours | 24 Hours | 48 Hours |
| DMF | Insoluble | Swells | Swells | Almost |
| | | | | Soluble* |
| DMSO | Swells | Swells | Swells | Almost |
| | | | | Soluble* |
| Toluene | Swells | Swells | Almost | Almost |
| | | | Soluble* | Soluble* |
| Methylene | Swells | Swells | Swells | Swells |
| Chloride | | | | |
| Ethanol | Insoluble | Insoluble | Insoluble | Insoluble |
| Ethyl Acetate | Insoluble | Insoluble | Insoluble | Insoluble |
| Water | Insoluble | Insoluble | Insoluble | Insoluble |
| Petroleum | Insoluble | Insoluble | Insoluble | Insoluble |
| Ether | | | | |
| Acetone | Insoluble | Insoluble | Insoluble | Insoluble |
| THF | Insoluble | Swells | Swells | Swells |
| Diethyl Ether | Insoluble | Insoluble | Insoluble | Insoluble |

*Solubility increases with increasing temperature

While the parent PAA is soluble in water and insoluble in non-polar organic solvents, the grafting copolymer showed substantial solubility in organic solvents such as toluene consistent with the amphiphilic nature of the copolymer product. The solubility of the cross-linker copolymer showed higher solubility in hydrophobic non-polar solvents than the hydrophilic polar solvents. In hydrophobic-non polar solvents the polymer swells with time but remains almost unchanged for hydrophilic-polar solvents. This indicates that the outer shell of the polymer is more hydrophobic in nature and therefore is most likely hydrophobic poly(t-butyl acrylate).

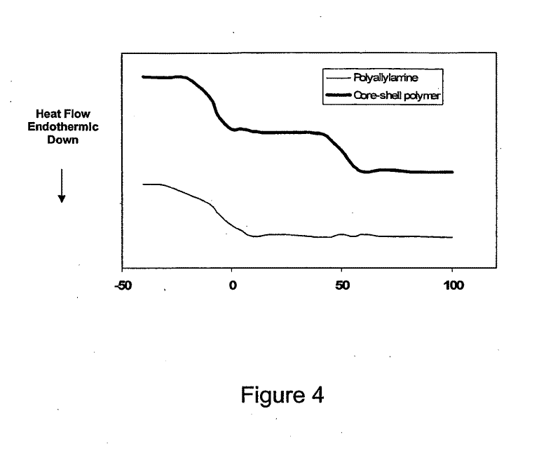

The DSC thermogram of the PAA and the core-shell material is shown in FIG. 4. In the first thermogram two glass-transition temperatures (T_{g}'s) are clearly observed, demonstrating the existence of two domains in the in same polymeric sample. The lower transition temperature appears at approximately −1.0° C. corresponds to the T_{g} of PAA. The T_{g} of pure PAA is not exact and ranges between −10.0° C. to 5.0° C., depending on the sample and its environment. The higher transition temperature appears at approximately 55° C. which is close to the T_{g} of poly(t-butyl acrylate), which is around 50.0° C. The emulsion made core-shell material thus depicts two transitions at −1.0° C. and 55° C. corresponding to the two components of the graft copolymer, the PAA and t-BA respectively.

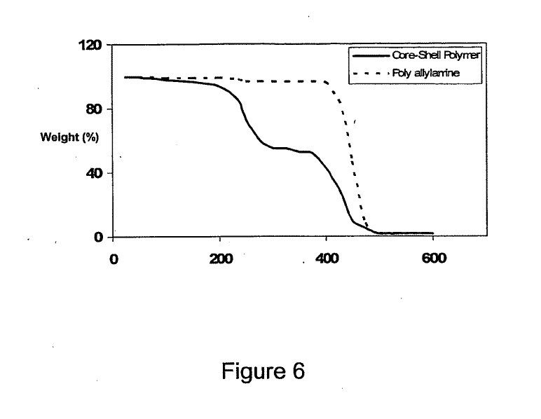

For the core-shell polymer, the thermogravimetric analysis (TGA) curve in FIG. 6 illustrates a two stage decomposition corresponding to the two phases of the polymer. The pure PAA polymer curve illustrates a decomposition temperature at approximately 470° C. The copolymer shows a first decomposition at approximately 260° C. and a second decomposition at 430° C. By comparing both curves, it can be concluded that first decomposition of polymer indicates the decomposition of the acrylate shell which melts around 245° C., whereas the second decomposition temperature demonstrates the decomposition of the PAA core. From the TGA analysis the first mass loss at 260° C. corresponds to an approximate 50% weight loss and the remaining 50% weight is lost by 430° C. This results shows that both the core and the shell of the polymer composition of the present invention constitute 50% of the total polymer weight.

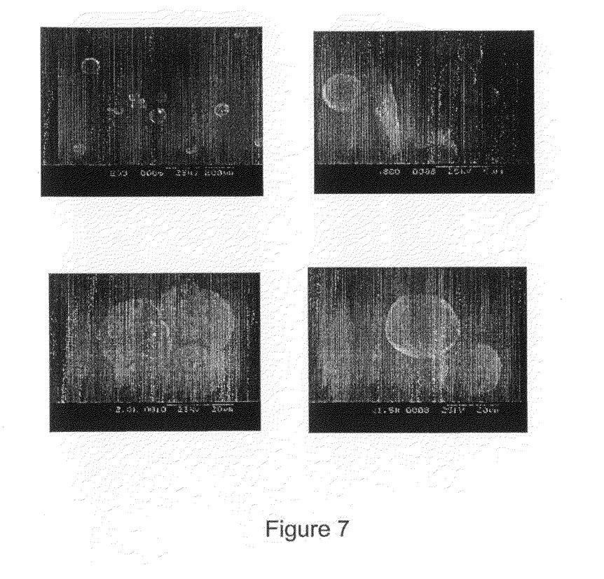

The morphology of the core-shell material is determined by scanning electron microscopy (SEM). The SEM micrographs of FIG. 7 show that grafting reaction of PAA by t-BA by inverse emulsion technique generates spheres of different size as shown in FIG. 7. From the micrographs, it is also evident that all the particles are not exactly spherical and some of the particles aggregated together over time. The aggregation of the particles generates some rough and peculiar surface patterns FIG. 7. This phenomenon of aggregation can be attributed to inter particle cross-linking during polymerization, as well as due to the physical linking in between the particles due to the presence of ester groups. Moreover the absence of surface charges on the particles may also lead to the agglomeration.

The TEM images visibly show the core-shell particle morphologies. The size of the particles ranges from the nanometer to micron level. The core structure is embedded in a thin shell of cross-linked acrylate. From the emulsion recipe and nature of the present invention, it can be concluded that most of the core is packed with water and is functionalized by amine groups.

In one embodiment of the present invention, the core-shell particles formed via the processes disclosed herein have average diameters of about 50 to about 1,000 nm, or from about 75 to about 750 nm, or from about 100 to about 500 nm, or even from about 250 to about 450 nm.

The results demonstrate that inverse emulsion polymerization can be used in conjunction with a water soluble initiator to generate a core-shell material with a hydrophilic core and a hydrophobic shell. A graft copolymer generated using such a reaction scheme can be manipulated to yield a core-shell structure. The functionalized amine group inside the hollow aqueous core permits encapsulation of drugs, cells, enzymes, fluorescent dyes (e.g., Eosin Y), dyes, nanoparticles (e.g., magnetic nanoparticles), and other biomaterials. Apart from biomedical applications these materials may be used in wide ranging applications such as sensing materials, catalysis, coating materials, filtration unit etc.

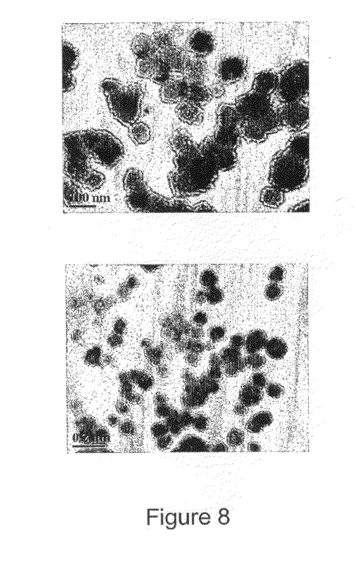

An amphiphilic graft copolymer according to the present invention has high grafting percentage and grafting ratio as is shown in Table 2. This indicates that most of the polyamine chains participate in the initiation process and are grafted by the t-butyl acrylate chains. The grafting efficiency, which indicates the amount of t-butyl acrylate monomers in the resulting copolymer, is consistent with a thin cross-linked shell structure shown in the TEM images (FIG. 8).

The formation of inverse emulsion can be exploited to synthesize amphiphilic copolymers from the water oil interface by a water-borne initiating system and oil soluble monomer, as shown in Scheme 1. Although not whishing to be bound solely thereto, a plausible reaction mechanism is shown in Scheme 2. The water phase of the inverse emulsion contains the initiator molecule hydrogen peroxide (H₂O₂) and the polyamine, which interacts initially to form the radical on the polyamine chain. The radical center on the polyallylamine can be either on the nitrogen or on the hydrocarbon backbone (through proton abstraction). This macroradical subsequently propagates the polymerization from the water/oil interface by grafting the t-butyl acrylate monomer. The propagation of the polymer proceeds from the interface towards the oil phase to generate t-butyl acrylate grafted polyallylamine. The water borne initiating system and oil phase propagation ensures the amphiphilic characteristics of the resulting graft copolymer. The use of a di-functional crosslinker molecule in the present invention leads to the formation of amphiphilic graft copolymer having a core-shell architecture. Ethylene diacrylate present in oil phase cross-links the growing t-butylacrylate chains around the interface of the W/O emulsion to form the hydrophobic shell structure within which the aqueous core functionalized with amine groups are embedded (see Scheme 3). The formation of core-shell structure from the amphiphilic graft copolymer further substantiates the mechanism of the interfacial character of the polymerization.

In one embodiment of the present invention, the products produced thereby are not solely limited to core-shell structures. Rather, any of the structures illustrated in FIG. 1 can be formed by varying/controlling the surfactant concentration in the emulsion in which polymerization is carried out. Alternatively, control of the polymerization reaction conditions and/or monomer composition can yield polymer structures in addition to the core-shell structures discussed above.

Although the invention has been described in detail with particular reference to certain embodiments detailed herein, other embodiments can achieve the same results. Variations and modifications of the present invention will be obvious to those skilled in the art and the present invention is intended to cover in the appended claims all such modifications and equivalents.

We claim:

1. A method for initiating radical polymerization of at least one monomer composition, the method comprising the steps of:
supplying at least one monomer charge; and
initiating radical polymerization of the at least one monomer charge via a hydrogen peroxide initiator and at least one polyamine co-initiator,
wherein the method is carried out in an inverse-microemulsion, the inverse-microemulsion being a water/oil emulsion.

2. The method of claim 1, wherein the at least one polyamine co-initiator is a polyallylamine.

3. The method of claim 1, wherein the at least one monomer is selected from one or more acrylate monomers, vinyl alcohol monomers, vinyl ether monomers, and carbonate monomers.

4. The method of claim 3, wherein the at least one monomer comprises tert-butyl acrylate.

5. A radical-initiator polymerization system comprising:
at least one monomer;
a hydrogen peroxide initiator; and
at least one co-initiator,
wherein the at least one co-initiator is a polyamine co-initiator and wherein polymerization is carried out in an inverse-microemulsion, the inverse-microemulsion being a water/oil emulsion.

6. The system of claim 6, wherein the at least one polyamine co-initiator is a polyallylamine.

7. The system of claim 6, wherein the at least one monomer is selected from one or more acrylate monomers, vinyl alcohol monomers, vinyl ether monomers, and carbonate monomers.

8. The system of claim 7, wherein the at least one monomer comprises tert-butyl acrylate.

9. A core-shell polymer composition produced by the method of claim 1.

10. The composition of claim 9, wherein the interior of the core-shell polymer contains at least one compound selected from drugs, cells, enzymes, fluorescent dyes, dyes, nanoparticles, or combinations of two or more thereof.

11. The composition of claim 9, wherein the core-shell polymer is an amphiphilic polymer.

12. The composition of claim 10, wherein the core-shell polymer contains at least one type of metal nanoparticles within its core.

13. The composition of claim 10, wherein the at least one type of metal nanoparticle is magnetic.
